(19) United States
(12) Patent Application Publication
(10) Pub. No.: US 2026/0306181 A1
(43) Pub. Date: Oct. 8, 2026

(54) SYSTEMS AND METHODS FOR SIMULATING VASCULAR SURGERY

(71) Applicant: Flyfruit Medtech Inc, Tustin, CA (US)

(72) Inventors: Heyan DONG, Santa Ana, CA (US); Xiangyi LI, Santa Ana, CA (US)

(21) Appl. No.: 19/637,435

(22) Filed: Apr. 2, 2026

Related U.S. Application Data
(60) Provisional application No. 63/782,408, filed on Apr. 2, 2025.

(51) Int. Cl. A61B 34/10; G09B 23/30

(52) U.S. Cl. CPC A61B 34/10; G09B 23/303; A61B 2034/105

(57) ABSTRACT

A vascular surgery simulator device, which includes a console simulation component, a display component, a blood vessel simulation component and a computer host, and the computer host data is connected to the console simulation component, the display component and the blood vessel simulation component. It is characterized in that the blood vessel simulation component includes a camera, a fill light, a vortex line trough plate, and an outer box. The inner bottom of the outer box is connected to the vortex line trough plate, and the inner top of the outer box is connected to the camera and the fill light. The vortex line through plate is provided with a vortex-shaped simulated blood vessel. The vascular surgery simulator device can simulate the key contents of interventional surgery, exercise the hands of interns, and allow interns to repeatedly practice necessary procedures such as surgical procedures and use of surgical instruments.

### CROSS REFERENCE TO RELATED APPLICATIONS

This application claims the benefit of U.S. Prov. Pat. App. No. 63/782,408, filed Apr. 2, 2025; the above application is incorporated by reference in its entirety.

### TECHNICAL FIELD

The embodiments described herein are generally directed to a surgical simulator device, and in particular to a vascular surgery simulator device.

### BACKGROUND

The requirements for the operation process of vascular interventional surgery are very strict. It requires rich experience, aided by angiography to confirm the location of a lesion and select appropriate access instruments and appropriate interventional treatment equipment according to the condition of the lesion. Unlike some other surgeries, vascular interventional surgery is not aided by three-dimensional impressions. During the operation, the interventional surgery must be completed by skillfully performing operations such as reaching the lesion location, angiography, and pressurizing the treatment equipment. Novice doctors and interns in vascular surgery need extensive training to master the overall process of interventional surgery and the use of related instruments. However, the number of donated cadavers in the country and the existing surgical operation requirements of hospitals cannot meet the training needs of novice doctors and interns. Novice doctors and trainees lack a device that allows them to repeatedly practice vascular interventional procedures.

### SUMMARY

Accordingly, systems, methods, and non-transitory computer-readable media are disclosed to a surgical simulator device.

According to one aspect, a vascular surgery simulator device, which includes a console simulation component, a display component, a blood vessel simulation component and a computer host, and the computer host data is connected to the console simulation component, the display component and the blood vessel simulation component. It is characterized in that the blood vessel simulation component includes a camera, a fill light, a vortex line trough plate, and an outer box. The inner bottom of the outer box is connected to the vortex line trough plate, and the inner top of the outer box is connected to the camera and the fill light. The vortex line through plate is provided with a vortex-shaped simulated blood vessel. The vascular surgery simulator device can simulate the key contents of interventional surgery, exercise the hands of interns, and allow interns to repeatedly practice necessary procedures such as surgical procedures and use of surgical instruments.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever. In addition, any of the methods described above and elsewhere herein, may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment;

FIG. 3 is an overall schematic diagram of an embodiment of the present invention;

FIG. 4 is an overall schematic diagram of a spiral trunking plate or vortex line trough plate according to an embodiment, showing the spiral grooves in the surface of the plate;

FIG. 5 is an overall schematic diagram of the operation keyboard according to an embodiment;

FIG. 6 is an overall schematic diagram of the outer box according to an embodiment; and

FIG. 7 is an overall schematic diagram of the outer box according to an embodiment.

### DETAILED DESCRIPTION

In an embodiment, systems, methods, and non-transitory computer-readable media are disclosed for a surgical simulator device.

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. System Overview

### 1.1. Infrastructure

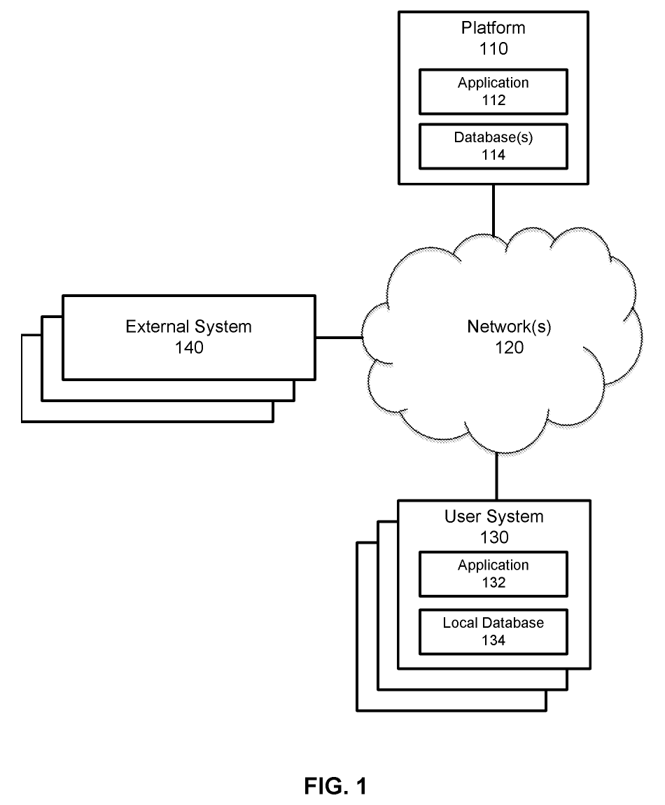

FIG. 1 illustrates an example infrastructure in which one or more of the disclosed processes may be implemented, according to an embodiment. The infrastructure may comprise a platform 110 (e.g., one or more servers) which hosts and/or executes one or more of the various processes (e.g., methods or functions, implemented as software modules) described herein. Platform 110 may comprise dedicated servers, or may instead be implemented in a computing cloud, in which the resources of one or more servers are dynamically and elastically allocated to multiple tenants based on demand. In either case, the servers may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 130 may comprise or be communicatively connected to a client application 132 and/or one or more local databases 134.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. It should be understood that platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from user system(s) 130 and/or external system(s) 140 and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes (e.g., methods and functionality), storage, and/or the like, described herein. For example, in such an embodiment, a client application 132, executing on one or more user system(s) 130, may interact with a server application 112 executing on platform 110 to execute one or more or a portion of one or more of the various process(es) described herein.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules comprising instructions that implement one or more of the processes (e.g., methods or functions) described herein.

### 1.2. Example Processing Device

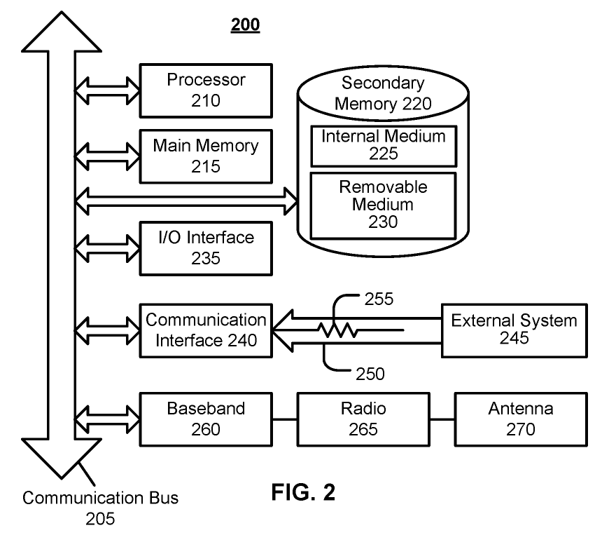

FIG. 2 is a block diagram illustrating an example wired or wireless system 200 that may be used in connection with various embodiments described herein. For example, system 200 may be used as or in conjunction with one or more of the processes (e.g., to store and/or execute the software), including any methods or functions, described herein, and may represent components of platform 110, user system(s) 130, external system(s) 140, and/or other processing devices described herein. System 200 can be any processor-enabled device (e.g., server, personal computer, etc.) that is capable of wired or wireless data communication. Other processing systems and/or architectures may also be used, as will be clear to those skilled in the art.

System 200 may comprise one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 210. Examples of processors which may be used with system 200 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 210 may be connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 may comprise main memory 215. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Python, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 200 may comprise secondary memory 220. Secondary memory 220 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 200. The computer software stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may include an internal medium 225 and/or a removable medium 230. Internal medium 225 and removable medium 230 are read from and/or written to in any well-known manner. Internal medium 225 may comprise one or more hard disk drives, solid state drives, and/or the like. Removable storage medium 230 may be, for example, a magnetic tape drive, a compact disc (CD) drive, a digital versatile disc (DVD) drive, other optical drive, a flash memory drive, and/or the like.

System 200 may comprise an input/output (I/O) interface 235. I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 200 may comprise a communication interface 240. Communication interface 240 allows software to be transferred between system 200 and external devices (e.g. printers), networks, or other information sources. For example, computer-executable code and/or data may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 240 is generally in the form of electrical communication signals 255. These signals 255 may be provided to communication interface 240 via a communication channel 250 between communication interface 240 and an external system 245 (e.g., which may correspond to an external system 140, an external computer-readable medium, and/or the like). In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code is stored in main memory 215 and/or secondary memory 220. Computer-executable code can also be received from an external system 245 via communication interface 240 and stored in main memory 215 and/or secondary memory 220. Such computer-executable code, when executed, enable system 200 to perform the various process(es) of the disclosed embodiments as described elsewhere herein.

In an embodiment that is implemented using software, the software may be stored on a computer-readable medium and initially loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes described elsewhere herein.

System 200 may comprise wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is communicatively coupled with processor(s) 210, which have access to memory 215 and 220. Thus, software can be received from baseband processor 260 and stored in main memory 210 or in secondary memory 220 or executed upon receipt. Such software, when executed, can enable system 200 to perform the various process(es) of the disclosed embodiments.

### 1.3. Vascular Surgery Simulator Device

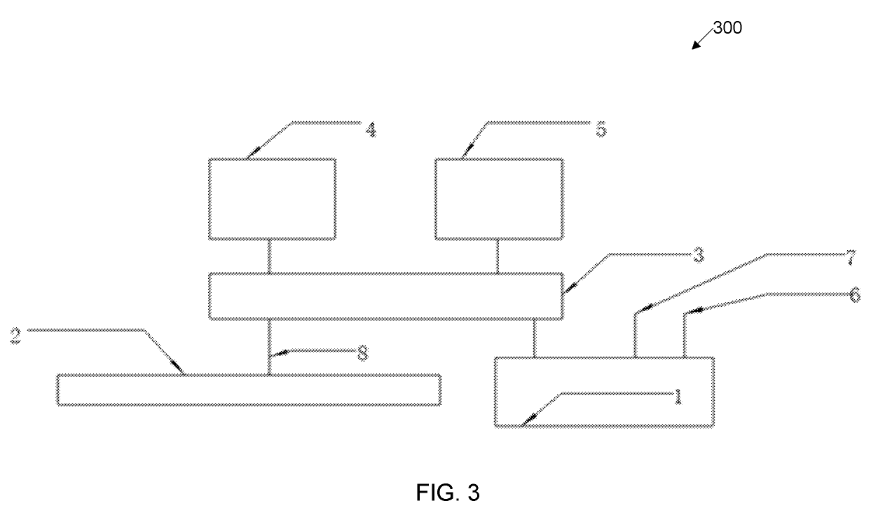

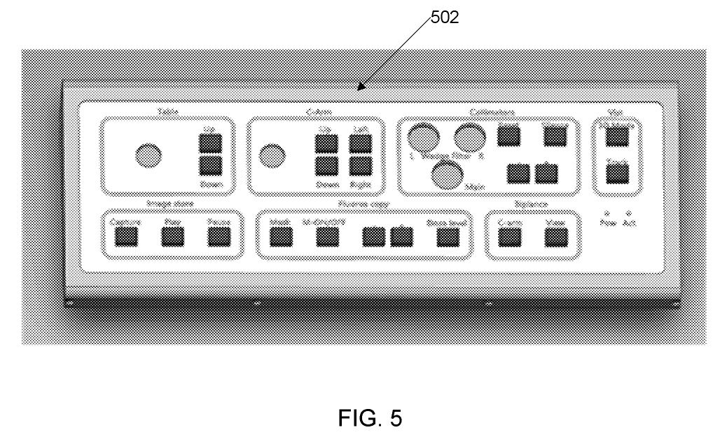

FIGS. 3-7 illustrate various aspects of a vascular surgery simulator device 300, in accordance with one embodiment. As can be seen, device 300 includes an operating console simulation components (1), blood vessel simulation components (2), computer host (3), image display area (4), operation display area (5), pressure pump interface (6), contrast agent interface (7), data cable (8), Camera (9) Vortex line trough plate (10), outer box (11), and photoelectric sensor components (12). These components will be explained in more detail below.

The computer host (3) is connected to the console simulation component (1), the display component (4), the display area (5) and the vascular simulation component (2). It also includes a software system, which can be implemented as application 112 and/or 132 as described below. The software system realizes the conversion of equipment data, analyzes the images captured by a vascular simulator camera (9) and the data transmitted by various sensors through algorithms, and compares them frame by frame with the uploaded standard surgical videos. The uploaded surgical videos may include interventional surgery imaging data (videos) from cases involving pulmonary arteries, coronary arteries, intracranial vessels, and peripheral vessels. Such vessels can be simulated using the vascular surgery simulator device, for example by the combination of the user performing a procedure on the simulated blood vessel while viewing an uploaded video of the same procedure in a patient, or while viewing images or feedback generated by the software system. The uploaded imaging data, e.g., videos from actual vascular surgery procedures, can be used by the software system to generate digital models for the simulated vascular surgery procedure, for example digital models of blood vessels, access routes, vessel tortuosity angles, or the extent of lesion stenosis and occlusion for different vascular conditions and treatment modalities.

The image display area (4) and operation display area (5) display the simulation status to determine whether the actual action meets the requirements of the standard surgical procedure. For example, the simulation may quantitatively evaluate key parameters during the simulated surgical procedure, such as contrast agent dosage, injection pressure, delivery distance, or the suitability of instrument selection. The software system may compare the observed values against those of standard or desired surgical workflows to provide an assessment of the physician's operational standardization, proficiency, and decision-making capabilities. In embodiments, the observed values are obtained by the software system from the video imaging of the simulated procedure obtained using the camera (9), and the values for standard or desired workflows are obtained by the software system from the uploaded procedure videos. Additionally, the software system may use the comparison between observed values and standard or desired workflows to generate real-time or post-operative feedback or comments on steps performed by the physician during the simulated vascular surgery procedure. Such feedback or comments can help physicians identify operational deviations and optimize procedural approaches, ultimately enhancing overall interventional surgery skills and clinical decision-making abilities.

The contrast agent interface (7) can be used to simulate the injection of a contrast agent for imaging in a patient. In some embodiments, the camera is a visible-spectrum camera. In some embodiments, non-visible wavelengths such as infrared, near-infrared, or UV are not imaged. In some embodiments, the vortex-shaped simulated blood vessel does not emit light.

Computer host (3) can be implemented as a user system 130, i.e., it can be local within the simulation environment, or it can be implemented as a platform 110, i.e., not local or within the simulation environment. In other embodiments, the functions, described in more detail below of computer host (3) can be implemented partially on a platform 110 and partially on a user system 130.

An advantage of having a platform 110 perform some or all of the functions of computer host (3) is that potentially platform 110 can control multiple systems 300. At the very least, data from the simulations incurring in multiple simulation locations can be uploaded to a platform 110 for analysis. This would allow the aggregation of data across simulations that can then be analyzed, e.g., with artificial intelligence and machine learning (AI/ML) to identify common errors, patterns, etc.

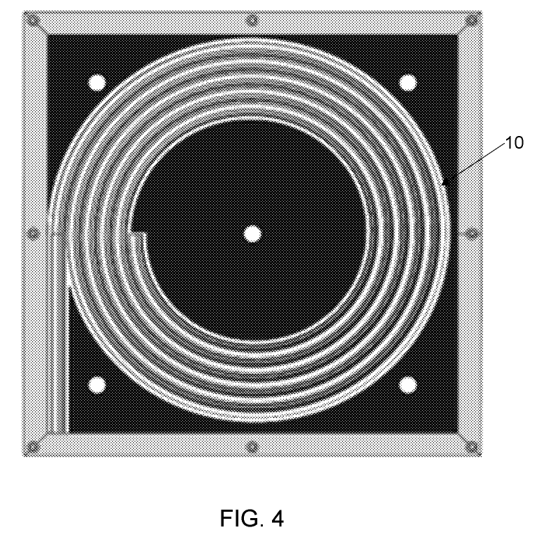

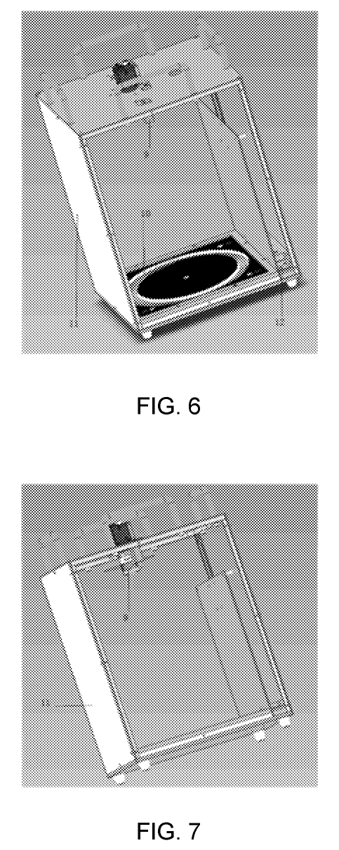

As illustrated in FIGS. 6 and 7, the simulation component (2) can comprise a housing (11). Within the housing (11) there can be a vortex line trough plate (10) that is used to simulate the surgery. The vortex line trough plate (10) is provided with a vortex-shaped simulated blood vessel. As illustrated in FIG. 4, the vortex-shaped simulated blood vessel can be formed into disk shape and configured as a series of concentric circles, but it will be understood that other configurations are possible.

In certain embodiments, the simulated blood vessel trough plate (10) is equipped with a pressure sensor, a flow sensor and a regulating valve. This allows for the simulation of surgical conditions as described below.

Housing (11) can also comprise a camera (9) and fill light (not shown), which can for example be ring fill light and a photoelectric sensor (12). The inside of the housing (11) can be coated with a non-reflective coating.

The operating console simulation component (1) can comprise an external simulation component (not shown) and an operation keyboard 502. The external simulation component can include a contrast agent interface (7) and a pressure pump interface (6). The contrast agent interface (7) and the pressure pump interface (6) are connected to the operation keyboard 502. The operation keyboard 502 can comprise buttons, rockers, switches, dials, and other inputs. The inputs can be configured to control page enlargement and reduction, as well as visual rotation on display area (5) as described below. In the embodiment illustrated in FIG. 3, the display area can include an image display area (4) and an operation display area (5).

The operation keyboard 502 is used to simulate the operation process of inspection items used before, during and after surgery, such as bed position, visual angle control, video page playback speed control, image inspection, etc. For example, button settings can realize functions such as page zoom in and out, and joystick settings can realize visual rotation functions.

The data connection between the computer host (3) and the console simulation component (1), the display area(s), and the blood vessel simulation component (2) can be via wired or wireless connections, as described with respect to FIG. 2.

In embodiments, “vortex” herein can refer to a spiral shape, such that a vortex line trough plate can be a plate that contains grooves or troughs in its surface that form a spiral as shown in FIG. 4. Such a spiral can result in a pattern of concentric rings of grooves or troughs in the surface of the vortex line trough plate, and such a plate can form the floor of the outer box that contains the simulated blood vessel as shown in FIG. 6. A “vortex-shaped simulated blood vessel” can be a tube or channel structure with a spiral shape, such that the tube or channel forms concentric circles as a spiral, with an opening of the tube or channel in the outer ring of the spiral; surgical instruments such as catheter-based instruments can be inserted through such an opening. Such a spiral configuration is shown in FIG. 4. In some embodiments, the vortex-shaped simulated blood vessel contains a single flow path through the vessel. In some embodiments, the vascular surgery simulator device models a single blood vessel.

In some embodiments, the vortex-shaped simulated blood vessel is integral to the vortex line trough plate. For example, it can be a channel in the plate formed from, in, or by the spiral grooves on the surface of the plate. In some embodiments, the vortex-shaped simulated blood vessel is placed on or in, or overlaid on or in, the spiral grooves on the surface of the vortex line trough plate. For example, in some embodiments, the vortex-shaped simulated blood vessel can be a tube that is positioned on the spiral grooves (which can also be termed vortex line troughs) on the surface of the vortex line trough plate, such that the tube also takes on a spiral configuration, overlapping the pattern made by the spiral grooves on the surface of the vortex line trough plate. In some embodiments, the spiral grooves on the surface of the vortex line trough plate, forming a concentric circular pattern, can serve a guide for the tube of the vortex-shaped simulated blood vessel, with the cross-section of the tube set in the groove. In some embodiments, a vortex-shaped simulated blood vessel formed from or on the grooves of a vortex line trough plate is held in place on the vortex line trough plate and does not move out of place, for example when contacted by a surgical instrument. In some embodiments, the simulated blood vessel has a coating on its inner surface. In some embodiments, the simulated blood vessel does not have a coating on its inner surface.

The configuration of the simulated blood vessel herein, particularly the vortex-shaped simulated blood vessel, including its shape, diameter, surface characteristics such as smoothness, and sensors such as mechanical force sensors and conventional camera imaging, allows actual surgical instruments to be used when the surgical simulator system is used, rather than the cut, shortened, bent, or otherwise not actual instruments often used in surgical simulators. This ensures that the positioning of the instruments within the blood vessels, and the experience of the user, better approximate that of real surgery. Any surgical instrument used in vascular surgery procedures, for example any surgical instrument that is used inside a blood vessel, may be used with the vascular surgery simulator device. Surgical instruments that may be used with the vascular surgery simulator device may include, for example: catheters such as diagnostic catheters, guide catheters, microcatheters, balloon catheters, ablation catheters, aspiration catheters, and infusion catheters; guidewires such as standard guidewires, hydrophilic guidewires, and support guidewires; delivery systems such as stent delivery systems, coil delivery systems, graft delivery systems, and embolic agent delivery systems; imaging devices such as intravascular ultrasound (IVUS) catheters, optical coherence tomography (OCT) catheters, and angioscopes; therapeutic devices such as atherectomy devices, thrombectomy devices, snares, retrieval devices, and biopsy forceps; occlusion devices such as balloons and vascular plugs; measurement devices such as pressure wires and flow measurement catheters; or other surgical instruments used in vascular procedures. In some embodiments, the surgical instrument is a catheter. In some embodiments, the surgical instrument is a balloon catheter. In some embodiments, the surgical instrument is an ablation catheter. In some embodiments, the surgical instrument is a catheter that deploys a stent. In some embodiments, the surgical instrument is an imaging device. In some embodiments, the surgical instrument is not an imaging device.

As such, a vascular surgery simulator device herein can comprise a surgical instrument capable of being used in a human vascular surgery procedure, or a vascular surgery simulation system can comprise a vascular surgery simulator device described herein and a surgical instrument capable of being used in a human vascular surgery procedure. A method of simulating a vascular surgery procedure that uses a vascular surgery simulator device herein can use a surgical instrument capable of being used in a human vascular surgery procedure. In some embodiments, the surgical instrument is inserted into a simulated blood vessel described herein to simulate a vascular surgery procedure. In some embodiments, the surgical instrument is inserted into the end of a simulated blood vessel described herein. In some embodiments, the surgical instrument is advanced along a simulated blood vessel described herein until it reaches the location of the simulated vascular surgery procedure inside the simulated blood vessel, for example a simulated lesion. In some embodiments, the user manipulates the surgical instrument inside the simulated blood vessel to simulate the vascular surgery procedure in the same way that they would manipulate the surgical instrument in a blood vessel of a patient when performing the actual vascular surgery procedure being simulated. In any of the above embodiments, the simulated blood vessel can be a vortex-shaped simulated blood vessel, or a tube or channel that forms concentric rings through a spiral shape. In any of the above embodiments, the surgical instrument can be one that is not an instrument used only for simulation, or the surgical instrument can be one that does not contain components not present in a surgical instrument used to perform the actual vascular surgery procedure being simulated. Such components can include sensors, actuators, lights, transmitters, transducers, magnets, dyes, or markers not present in a surgical instrument used to perform the actual vascular surgery procedure being simulated.

Any vascular surgery procedure may be simulated using the vascular surgery simulator device. Vascular surgery procedures that can be simulated may include any that involve placing or using a surgical instrument inside a blood vessel. Such procedures may include, for example: catheter-based interventions such as angioplasty, balloon dilation, and atherectomy; device placement procedures such as stent deployment, coil embolization, graft placement, and vena cava filter insertion; ablation procedures such as radiofrequency ablation, cryoablation, and laser ablation; diagnostic procedures such as angiography, intravascular ultrasound (IVUS), optical coherence tomography (OCT), and fractional flow reserve (FFR) measurement; thrombectomy and embolectomy procedures; vessel repair procedures such as suturing, anastomosis, and wound repair; biopsy procedures; drug delivery procedures; vessel occlusion procedures; arteriovenous fistula creation; endovascular aneurysm repair (EVAR); carotid artery stenting; peripheral vascular interventions; coronary interventions; neurovascular interventions; or other vascular surgery procedures. In some cases, the simulator device may be used to practice navigation of surgical instruments through tortuous vasculature, deployment of therapeutic devices at target lesion sites, and coordination of imaging with interventional maneuvers. The vascular surgery simulator device may also be configured to simulate procedures involving pulmonary arteries, coronary arteries, intracranial vessels, and peripheral vessels

### 2. Process Overview

Embodiments of processes for a surgical simulator device will now be described in detail. It should be understood that the described processes may be embodied in one or more software modules that are executed by one or more hardware processors (e.g., processor 210), for example, as a software application (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the software application are executed by platform 110 and other portions or modules of the software application are executed by user system(s) 130. The described processes may be implemented as instructions represented in source code, object code, and/or machine code. These instructions may be executed directly by a hardware processor(s) 210, or alternatively, may be executed by a virtual machine operating between the object code and hardware processor(s) 210. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

Furthermore, while the processes described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

Under the control of the software described above, the pressure-time curve and flow-time curve data of medicines such as heparin, contrast media, and equipment such as pressure pumps entering a blood vessel receive actual data through pressure sensors and flow sensors included within vortex-shaped simulated blood vessel included in plate 10 as described above. Flow and pressure in vortex-shaped simulated blood vessel are adjusted through regulating valves to make the actual data close to the pressure-time curve and flow-time curve data after medicines such as heparin, contrast media, and equipment such as pressure pumps enter real blood vessels. Such regulating valves can be located at any point along the length of the simulated blood vessel, or they can be located at either end of the simulated blood vessel. The function of simulating medicines such as heparin, contrast media, and equipment such as pressure pumps is realized, and inspection items and parameters such as heparin dosage and contrast medium dosage are realized, as is simulation of surgical items and aspects such as the degree of balloon dilation.

Thus, the trainee can navigate a catheter or simulated catheter through the vortex-shaped simulated blood vessel to simulate an actual vascular surgery, with realistic feedback. The entrance to the vortex-shaped simulated blood vessel can be located outside housing (11). The inside of the housing (11) should be relatively separated from the external environment and is a closed environment.

As the trainee moves the catheter through vortex-shaped simulated blood vessel, images can be displayed in display area of a catheter actually moving through the vasculature of an actual patient. Thus, blockages, problems, issues, etc., can be simulated in the images, and require the trainee to navigate them by moving the catheter within vortex-shaped simulated blood vessel. The contrast agent interface (7) and pressure pump interface (6) use the interface to simulate the flow and pressure data that need to be directly inserted into the contrast catheter and balloon during actual surgery. In this way, the flow and pressure data can be simulated through the interface simulation, which facilitates the scoring of the simulation training operation and avoids the actual insertion of instruments, contamination of the vortex line cauldron of the simulator, and poor detection.

Progress of the training catheter moving through vortex-shaped simulated blood vessel can be tracked using camera (9) aided by the fill light. The internal coating design of the housing (11) ensures that there is no reflection inside the box and does not affect the image transmission from camera (9).

Thus, system 300 can simulate the passage, position, sequence and other functions of multiple instruments in a blood vessel. Among them, catheters, guide wires, balloons, stents, etc. used in interventional procedures should be placed in the simulated vascular access.

The inner surface of vortex-shaped simulated blood vessel should be smooth and burr-free, allowing smooth passage of the training catheter. The size of the troughs in the board can adapt or can be adapted to the sizes of all catheters, guide wires, balloons, and stents currently on the market.

The photoelectric sensor component (12) of the vascular surgery simulator device 300 can initially be purged by a diaphragm air pump (not shown) to complete self-cleaning, which is convenient for repeated practice. The photoelectric sensor component (12) should be located at the entrance of the catheter into the vortex-shaped simulated blood vessel. The direction of rotation of the catheter, one of the surgical instruments, can be monitored via photoelectric sensor (12) and computer host (3).

Thus, a method for using the device 300 can include the steps of: turn on the hardware power switch, open the software, log in to the training account, and enter the training page. The corresponding simulation data provided by the contrast agent interface and the pressure pump interface detects the flow data and pressure data in the simulated blood vessel or device through the data sensor and pressure sensor, and controls the pressure data and flow data in the simulated blood vessel or device through the regulating valve to make it close to the corresponding simulation data provided by the contrast agent interface and the pressure pump interface. According to the standard surgical video displayed in the image display area, the operator simulates the surgical process on the simulated blood vessel by operating the keyboard and simulating preoperative, intraoperative and postoperative inspection items, and feeds the instruments in sequence according to the surgical requirements. Under the requirements of the standard surgical video prompts, the operator passes the detection instruments such as contrast catheters, Guide wires, catheters, etc. accurately find the location of the lesion, perform lesion assessment, and send treatment instruments such as balloons and stents to the designated lesion location to perform standard and effective treatment of the lesion. The simulated surgical process is displayed in the operation display area, and the system scores the operator's operation sequence, operation time, and operation standards.

The following examples illustrate how a user may use the vascular surgery simulator device in various training scenarios. These examples are provided for illustrative purposes and are not intended to limit the scope of the invention.

### Example 1: Coronary Angioplasty Training Scenario

In some embodiments, a trainee may use the vascular surgery simulator device to practice a coronary angioplasty procedure. The trainee may begin by turning on the hardware power switch and opening the software system on the computer host. After logging in to a training account, the trainee may select a coronary angioplasty training module from the available procedure options displayed in the image display area.

Once the training module is loaded, the system may display a standard surgical video in the image display area showing the expected procedure workflow. The trainee may then insert a guide catheter into the entrance of the vortex-shaped simulated blood vessel located outside the housing. Using the operation keyboard, the trainee may adjust the visual angle and page zoom as needed to observe the catheter position.

As the trainee advances the guide catheter through the vortex-shaped simulated blood vessel, the camera within the housing may capture the catheter movement, and corresponding angiographic images may be displayed in the operation display area. The trainee may navigate the catheter toward a simulated coronary lesion location. Upon reaching the target site, the trainee may use the contrast agent interface to simulate injection of contrast media for lesion visualization.

The trainee may then advance a guidewire through the guide catheter and across the simulated lesion. Following guidewire placement, the trainee may introduce a balloon catheter over the guidewire and position it at the lesion site. Using the pressure pump interface, the trainee may simulate balloon inflation to dilate the simulated stenosis. The pressure sensor and flow sensor within the simulated blood vessel may provide feedback data to the software system.

After balloon dilation, the trainee may deploy a stent at the lesion location by advancing a stent delivery system and releasing the stent. The software system may compare the trainee's actions against the standard surgical workflow and provide scoring based on operation sequence, operation time, instrument selection, contrast agent dosage, and injection pressure. Real-time or post-operative feedback may be displayed to help the trainee identify areas for improvement.

### Example 2: Peripheral Vascular Intervention Training Scenario

In some embodiments, a trainee may use the vascular surgery simulator device to practice a peripheral vascular intervention, such as treatment of a simulated peripheral artery blockage. The trainee may log in to the training account and select a peripheral vascular intervention module from the software system.

The image display area may present uploaded surgical video data corresponding to peripheral vessel interventions. The trainee may insert appropriate access instruments, such as a sheath and diagnostic catheter, into the vortex-shaped simulated blood vessel. The trainee may navigate the catheter through the simulated vasculature while observing progress in the operation display area.

Upon identifying the simulated peripheral lesion, the trainee may perform lesion assessment using the contrast agent interface to simulate angiography. The trainee may then select appropriate treatment instruments based on the simulated lesion characteristics. For example, the trainee may advance an atherectomy device to remove simulated plaque, followed by balloon angioplasty and stent placement if indicated.

Throughout the procedure, the software system may monitor the trainee's technique, including catheter manipulation, instrument sequencing, and use of the operation keyboard for visual adjustments. The regulating valve within the simulated blood vessel may adjust flow and pressure conditions to simulate realistic hemodynamic responses. Upon completion, the system may provide a comprehensive assessment of the trainee's performance.

### Example 3: Neurovascular Intervention Training Scenario

In some embodiments, a trainee may use the vascular surgery simulator device to practice neurovascular interventions involving navigation through tortuous intracranial vessels. The trainee may select a neurovascular intervention training module, which may load digital models of intracranial vessel anatomy including vessel tortuosity angles and lesion characteristics.

The trainee may begin by inserting a guide catheter into the vortex-shaped simulated blood vessel and carefully advancing it through the simulated tortuous pathway. The spiral configuration of the vortex-shaped simulated blood vessel may provide a realistic simulation of the winding nature of intracranial vasculature. The photoelectric sensor component may monitor the direction of catheter rotation as the trainee navigates through the simulated vessels.

Using a microcatheter and microguidewire, the trainee may navigate to a simulated intracranial lesion, such as an aneurysm or arteriovenous malformation. The trainee may practice deploying therapeutic devices, such as coils or flow diverters, at the target site. The camera and fill light within the housing may capture the instrument positions, and the software system may analyze the captured images frame by frame.

The software system may evaluate the trainee's ability to navigate through the tortuous simulated vasculature without causing simulated vessel injury, proper positioning of therapeutic devices, and coordination of imaging with interventional maneuvers. Feedback may be provided regarding the trainee's proficiency in handling delicate neurovascular instruments and decision-making capabilities in complex anatomical situations.

In each of the above examples, the vascular surgery simulator device may allow trainees to repeatedly practice the procedures using actual surgical instruments, thereby developing muscle memory and procedural familiarity in a controlled training environment.

### SPECIFIC EMBODIMENTS

Embodiment 1. A vascular surgery simulator device, including an operating table simulation component (1), a display component, a blood vessel simulation component (2) and a computer host (3). The computer host (3) is data connected to the operating console simulation component (1), the display component and the blood vessel simulation component (2). The blood vessel simulation component (2) includes a camera (9), a fill light, a vortex line trough plate (10), and an outer box (11). The inner bottom of the outer box (11) is connected to the vortex line trough plate (10). The inner top of the outer box (11) is connected to the camera (9) and the fill light. The vortex line trough plate (10) is provided with a vortex-shaped simulated blood vessel.

Embodiment 2. The vascular surgery simulator device of embodiment 1, characterized in that the console simulation component (1) includes an external simulation component and an operation keyboard, the external simulation component includes a contrast agent interface (7) and a pressure pump interface (6), the contrast agent interface (7) and the pressure pump interface (6) are connected to the operation keyboard, the operation keyboard is provided with buttons and a rocker, the buttons control page enlargement and reduction, and the rocker controls visual rotation.

Embodiment 3. The vascular surgery simulator device of embodiment 1, characterized in that the display component includes an image display area (4) and an operation display area (5).

Embodiment 4. The vascular surgery simulator device of embodiment 1, characterized in that the simulated blood vessel is provided with a pressure sensor, a flow sensor and a regulating valve.

Embodiment 5. The vascular surgery simulator device of embodiment 1, characterized in that a non-reflective coating is provided on the inside of the outer box.

Embodiment 6. The vascular surgery simulator device of embodiment 1, characterized in that the fill light is a ring-shaped fill light.

Embodiment 7. The vascular surgery simulator device of embodiment 1, characterized in that the spiral groove plate (10) is in the shape of a disk as a whole.

Embodiment 8. The vascular surgery simulator device of embodiment 1, characterized in that the data connection method of the computer host (3) to the console simulation component (1), the display component and the blood vessel simulation component (2) is a wired connection, and the console simulation component (1), the display component and the blood vessel simulation component (2) are all provided with data line jacks.

The above description and disclosed embodiments are provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B’s, multiple A’s and one B, or multiple A’s and multiple B’s.

What is claimed is:

1. A vascular surgery simulator device comprising a console simulation component, a display component, a blood vessel simulation component and a computer host.

2. The vascular surgery simulator device of claim 1, wherein the computer host is data connected to the console simulation component, the display component and the blood vessel simulation component.

3. The vascular surgery simulator device of claim 1, wherein the blood vessel simulation component comprises a camera, a fill light, a vortex line trough plate, and an outer box.

4. The vascular surgery simulator device of claim 3, wherein an inner bottom of the outer box is connected to the vortex line trough plate.

5. The vascular surgery simulator device of claim 3, wherein an inner top of the outer box is connected to the camera and the fill light.

6. The vascular surgery simulator device of claim 3, further comprising a vortex-shaped simulated blood vessel provided with the vortex line trough plate.

7. The vascular surgery simulator device of claim 1, wherein the console simulation component comprises an external simulation component and an operation keyboard; the external simulation component comprises a contrast agent interface and a pressure pump interface; the contrast agent interface and the pressure pump interface are connected to the operation keyboard; the operation keyboard comprises buttons and a rocker switch; the buttons control page enlargement and reduction; and the rocker switch controls visual rotation.

8. The vascular surgery simulator device of claim 1, wherein the display component comprises an image display area and an operation display area.

9. The vascular surgery simulator device of claim 6, further comprising a pressure sensor, a flow sensor and a regulating valve provided with the simulated blood vessel.

10. The vascular surgery simulator device of claim 3, wherein a non-reflective coating is present on the inside of the outer box.

11. The vascular surgery simulator device of claim 3, wherein the fill light is a ring-shaped fill light.

12. The vascular surgery simulator device of claim 3, wherein the vortex line trough plate comprises spiral grooves.

13. The vascular surgery simulator device of claim 12, wherein the spiral grooves are in the shape of a disk.

14. The vascular surgery simulator device of claim 1, wherein the computer host has a data connection to the console simulation component; the display component has a data connection to the blood vessel simulation component that is a wired connection; and the console simulation component, the display component and the blood vessel simulation component all contain data line jacks.
